(19) United States
(12) Patent Application Publication
(10) Pub. No.: US 2006/0035872 A1
(43) Pub. Date: Feb. 16, 2006

(54) Process for the preparation of 3-oximino steroids

(76) Inventors: Marco Villa, Milano (IT); Roberta Fretta, Torino (IT); Nicola Diulgheroff, Torino (IT)

Correspondence Address: KENYON & KENYON, ONE BROADWAY, NEW YORK, NY, 10004, US

(21) Appl. No.: 11/202,191

(22) Filed: Aug. 12, 2005

Related U.S. Application Data
(60) Provisional application No. 60/600,780, filed on Aug. 12, 2004.
(60) Provisional application No. 60/609,839, filed on Sep. 15, 2004.

(51) Int. Cl. A61K 31/56; C07J 41/00

(52) U.S. Cl. 514/169; 552/519

(57) ABSTRACT

The present invention provides a method of preparing norelgestromin or norgestimate by reacting the corresponding 3-oxosteroid precursor with hydroxylamine HCl and a base to obtain a reaction mixture forming norelgestromin or norgestimate; monitoring the anti/syn ratio of the norelgestromin or norgestimate produced in the reaction mixture; adding a base to the reaction mixture to neutralize acidity in the reaction mixture when a desired anti/syn ratio is detected; and isolating the norelgestromin or norgestimate. The present invention also provides a process allowing a control of the formation of the anti isomer and syn isomer of norelgestromin or norgestimate by reacting the corresponding 3-oxosteroid precursor with hydroxylamine HCl and a base to obtain a reaction mixture forming norelgestromin or norgestimate; regulating the acidity of the reaction mixture to adjust the anti/syn ratio of the norelgestromin or norgestimate produced in the reaction mixture; adding a base to the reaction mixture to neutralize acidity in the reaction mixture when a desired anti/syn ratio is detected; and isolating the norelgestromin or norgestimate.

This application claims the benefits of U.S. Provisional Application No. 60/600,780 filed Aug. 12, 2004 and U.S. Provisional Application No. 60/609,839 filed Sep. 15, 2004, the disclosures of which are incorporated by reference.

The present invention is related to a process for preparing norelgestromin or norgestimate.

### BACKGROUND

Norelgestromine, having the formula: 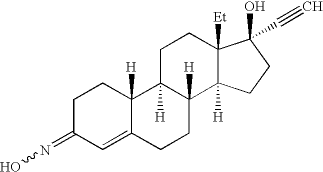
has the chemical name, D-17α-ethynyl-13β-ethyl-gon-4-en-17β-ol-3-one oxime; 17-ethynyl-17-ethynyl-17β-hydroxy-19-methylestr-4-en-3-one oxime; or 13-ethyl-17β-hydroxy-18,19-dinorpregn-4-en-20-yn-3-one-3-oxime. It is an active progestin largely responsible for the progestational activity that occurs in women following application of ORTHO EVRA®. It is also a primary active metabolite produced following oral administration of Norgestimate, having the formula: 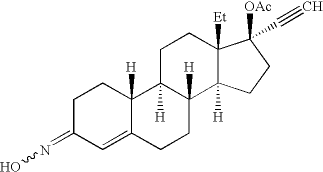
that has the chemical name, 17-acetoxy-13-ethyl-18,19-dinorpregn-4-en-20-yn-3-one-3-oxime, (17 α); or D-17β-acetoxy-13β-ethyl-17α-ethynyl-gon-4-en-3-one oxime. Norgestimate is the progestin component of the oral contraceptive products ORTHO-CYCLEN® and ORTHO-TRI-CYCLEN®.

3-Oximino steroids, such as norelgestromin and norgestimate, are classically prepared by reacting the corresponding 3-oxosteroids with NH₂OH.HCl in the presence of a base, usually pyridine, AcONa or NaOH. Both norelgestromin and norgestimate are mixtures of syn and anti isomers at the oxime position.

Preparation of compounds with structures very similar to norelgestromin and norgestimate are described in several examples of a number of papers and patents. For example, 17α-ethynyl-19-norandrost-4-en-17β-ol-3-one oxime was prepared by reacting 17α-ethynyl-19-norandrost-4-en-17β-ol-3-one with hydroxylamine hydrochloride in pyridine on a steam bath for two hours (see BE 718271; U.S. Pat. No. 3,532,689; U.S. Pat. No. 3,629,415). The solution was poured over a large amount of water and the precipitate thus formed was recovered by filtration. Recrystallization from methanol gave 17α-ethynyl-19-norandrost-4-en-17β-ol-3-one oxime.

Similarly 17β-acetoxy-13β-ethyl-17α-ethynyl-gon-4-en-3-one oxime was prepared by reacting 17β-acetoxy-13β-ethyl-17α-ethynyl-gon-4-en-3-one with hydroxylamine hydrochloride in pyridine in a steam bath for 45 min. The solution was cooled and poured over a large amount of water; the solid thus formed was recovered by filtration. Recrystallization from a dichloromethane/ethanol mixture gave 17β-acetoxy-13β-ethyl-17α-ethynyl-gon-4-en-3-one oxime with a yield of 89.6% (see U.S. Pat. No. 4,027,019; BE 844350).

U.S. Pat. No. 4,186,128 discloses a method for obtaining 3-oximino steroids by reacting the corresponding 3-pyrrolidyl enamine in the presence of hydroxylamine hydrochloride and sodium acetate to give the 3-oximino derivative; under these conditions the 3-keto-Δ⁴-derivative was not formed.

The US Pharmacopoeia sets a very strict anti/syn isomeric ratio of 1.27-1.78. There is, therefore, a need in the art for a process for preparing 3-oximino steroids that control the anti/syn isomeric ratio.

### SUMMARY OF THE INVENTION

One aspect of the invention is directed toward a method for preparing a 3-oximino steroid of the formula III 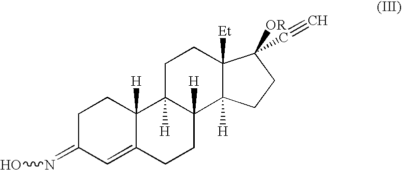
having an anti/syn ratio of about 0.5 to about 3, wherein R is H or acetyl, comprising the steps of:
a) combining the corresponding precursor 3-oxosteroid of formula IV 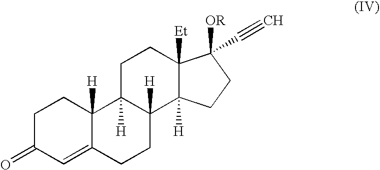
   wherein

R is either H or acetyl, and a polar organic solvent to obtain a suspension;
b) combining the suspension of step (a) with a first base and a hydroxylammonium salt, to obtain a reaction mixture;
c) maintaining the obtained reaction mixture until the anti/syn ratio of the obtained 3-oximino steroid of the formula III is of about 0.5 to about 3;
d) combining a second base with the reaction mixture obtained in step c) when the desired anti/syn ratio of the obtained 3-oximino steroid is detected; and
e) recovering the compound of formula III.
When R is H, said compound of the formula (IV) corresponds to Levonorgestrel, 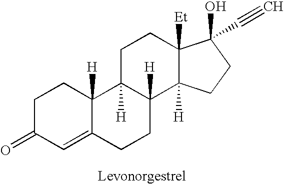
   and the obtained product of formula (III) corresponds to Norelgestromin.

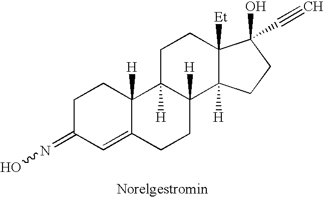

When R is acetyl, said compound of the formula (IV) corresponds to Levonorgestrel 17-acetate, 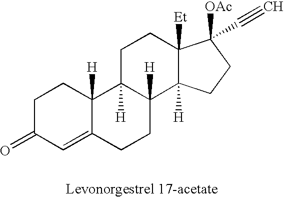
and the obtained product of formula (III) corresponds to Norgestimate.

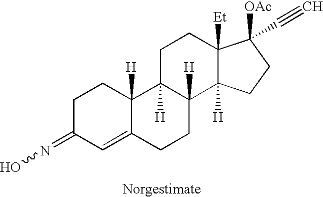

3-oximino steroid of the formula III prepared by the above process is obtained in high purity of about 95% to about 100%, by HPLC.

Norelgestromin prepared by the above process is obtained in high purity, wherein the level of Levonorgestrel is less than about 5%, by HPLC. Preferably, the level of Levonorgestrel is less than about 0.1%, by HPLC.

Norgestimate prepared by the above process is obtained in high purity, wherein both, Levonorgestrel 17-acetate and Norelgestromine, are contained in less than about 5%, by HPLC. Preferably, both, Levonorgestrel 17-acetate and Norelgestromine, are contained in less than about 2%, by HPLC.

Another aspect of the present invention is Norelgestromin that contains less than about 5% area by HPLC of Levonorgestrel.

A further aspect of the present invention is Norelgestromin that contains less than about 0.1% area by HPLC of Levonorgestrel.

Yet another aspect of the present invention is Noregestimate that contains less than about 5% area by HPLC of both Levonorgestrel 17-acetate and Norelgestromin.

Yet a further aspect of the present invention is Noregestimate that contains less than about 2% area by HPLC of both Levonorgestrel 17-acetate and Norelgestromin.

Preferably, the amount of the first base in step (b) is less than about 1.4 mole equivalents per mole equivalent of Levonorgestrel or of Levonorgestrel 17-acetate, more preferably of about 1.1 mole equivalents per mole equivalent of Levonorgestrel or of Levonorgestrel 17-acetate.

Preferably, the anti/syn ratio obtained by the above process is of about 2.08, by HPLC.

### DETAILED DESCRIPTION OF THE INVENTION

As used herein, the term “purity” relates to the amount of the desired product presented in the compound in question.

As used herein, the term “high purity” in reference to the 3-oximino steroid relates to a purity of about 95% to about 100%, by HPLC.

In the prior art, 3-oximino steroids are classically prepared using pyridine as a solvent of the reaction. The present invention can apply methanol instead, leading to a process of lower toxicity and cost that can be used in large scale.

Moreover, the procedure of the present invention provides a method for controlling the anti/syn isomer ratio of the 3-oximino steroid, a parameter which is required in order to meet the limitations of the US Pharmacopeia.

3-Oximino steroids of formula III are obtained by the process of the present invention in high yields and in high purity, such that no purification steps are usually required.

The present invention provides a method for preparing a 3-oximino steroid of the formula III 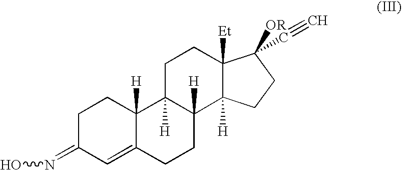
having an anti/syn ratio of about 0.5 to about 3, wherein R is H or acetyl, comprising the steps of:
a) combining the corresponding precursor 3-oxosteroid of formula IV 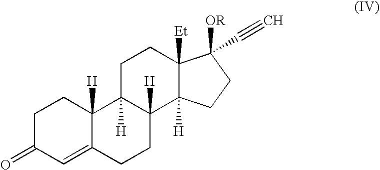
   wherein

R is either H or acetyl, and a polar organic solvent to obtain a suspension;
b) combining the suspension of step (a) with a first base and a hydroxylammonium salt, to obtain a reaction mixture;
c) maintaining the obtained reaction mixture until the anti/syn ratio of the obtained 3-oximino steroid of the formula III is of about 0.5 to about 3;
d) combining a second base with the reaction mixture obtained in step c) when the desired anti/syn ratio of the obtained 3-oximino steroid is detected; and
e) recovering the compound of formula III.

When R is H, said compound of the formula (IV) corresponds to Levonorgestrel, 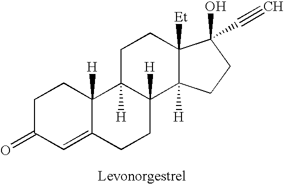
and the obtained product of formula (III) corresponds to Norelgestromin.

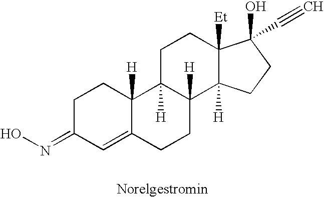

When R is acetyl, said compound of the formula (IV) corresponds to Levonorgestrel 17-acetate. 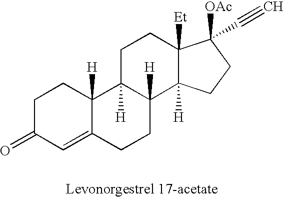
and the obtained product of formula (III) corresponds to Norgestimate.

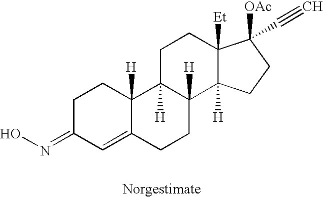

3-oximino steroid of the formula III prepared by the above process is obtained in high purity of about 95% to about 100%, by HPLC.

Norelgestromin prepared by the above process is obtained in high purity, wherein the level of Levonorgestrel is less than about 5%, by HPLC. Preferably, the level of Levonorgestrel is less than about 0.1%, by HPLC.

Norgestimate prepared by the above process is obtained in high purity, wherein both, Levonorgestrel 17-acetate and Norelgestromine, are contained in less than about 5%, by HPLC. Preferably, both, Levonorgestrel 17-acetate and Norelgestromine, are contained in less than about 2%, by HPLC.

The present invention further provides Norelgestromin that contains less than about 5% area by HPLC of Levonorgestrel, preferably, less than about 0. 1% area by HPLC of Levonorgestrel.

The present invention also provides Noregestimate that contains less than about 5% area by HPLC of both Levonorgestrel 17-acetate and Norelgestromin preferably, less than about 2% area by HPLC of both Levonorgestrel 17-acetate and Norelgestromin.

Preferably, Levonorgestrel is commercially available.

Preferably, Levonorgestrel 17-acetate is obtained by esterification of the 17-OH of Levonorgestrel.

The polar organic solvent used in step (a) is selected from the group consisting of straight or branched C₁₋₄ alcohol, ether, nitrile, amide, and sulfoxide. A preferred straight or branched C₁₋₄ alcohol is methanol, ethanol, 1-propanol, 2-propanol or t-butanol. Preferably, the ether is 1,4-dioxane. A preferred nitrile is acetonitrile. Preferably, the amide is dimethylformamide or dimethylacetamide. A preferred sulfoxide is dimethylsulfoxide. More preferably, the polar organic solvent is methanol.

The hydroxylammonium salt used in step (b) is selected from the group consisting of hydroxylamine hydrochloride, hydroxylamine sulphate and hydroxylamine phosphate. Preferably, the salt is hydroxylamine hydrochloride.

The preferred amount of hydroxylamine hydrochloride is of about 1.4 mole equivalent per mole of Levonorgestrel or of Levonorgestrel 17-acetate.

Suitable first base used in step (b) is selected from the group consisting of alkoxide, alkali hydroxide, carbonate of alkali metals and acetate of alkali metal. A preferred alkoxide is of sodium or potassium, more preferably, methoxide, ethoxide, propoxide or t-butoxide. Preferably, the alkali hydroxide is sodium hydroxide or potassium hydroxide. A preferred carbonate of alkali metal is sodium or potassium carbonate and bicarbonate. Preferably, the acetate of an alkali metal is sodium acetate. The most preferred base is sodium methoxide.

The order of combining the reacting substances in step (b) influences the purity of the final product. Preferably, the precursor 3-oxo steroid is combined with the first base, and only then, the hydroxylammonium salt is added.

The temperature in step (c) is preferably of about 20° C. to about 65° C.

The mixture of step (c) can be maintained for at least about 3 hours, preferably for about 3 to 16 hours.

3-Oximino steroid of formula III, wherein R is H or Ac, prepared by the above method is a mixture of anti and syn isomers at the oximo position. The initial ratio of the anti/syn is mainly determined by the rate of transformation of 3-oxosteroid of formula IV, wherein R is H or Ac, into the anti and syn isomers of 3-oximino steroid of formula III, wherein R is H or Ac, and then the anti/syn isomerization takes place until a constant value is reached at thermodynamic equilibrium.

The ratio of the anti/syn isomer of the 3-oximino steroid of formula III, wherein R is H or Ac, in step (c) can be controlled by variation of the acidity of the reaction medium by adding a suitable amount of the first base.

Preferably, the anti/syn ratio obtained by the above process is of about 2.08, by HPLC. The above ratio is obtained by addition of a suitable amount of the first base, preferably less than about 1.4 mole equivalents per mole equivalent of Levonorgestrel or of Levonorgestrel 17-acetate, and more preferably, of about 1.1 mole equivalents per mole equivalent of Levonorgestrel or of Levonorgestrel 17-acetate.

Preferably, when the above anti/syn ratio is obtained, it is fixed by addition of a second base to complete neutralization of the acidity, to block the catalytic effect of an acid on the anti-syn interconversion.

Preferably, the second base is the same as the first base; more preferably, the second base is sodium methoxide.

3-Oximino steroid is recovered by any methods known in the art, such as filtering the obtained suspension after the addition of the second base, to dispose impurities, such as NaCl, followed by adding the filtrate to cold water to induce precipitation of 3-oximino steroid.

The present invention is demonstrated with examples below. The scope of the invention, however, should be measured by the claims. Modifications, by a person skilled in the art, of the embodiments demonstrated in any of the examples are within the scope of the invention.

### EXAMPLES

### Example 1

### Preparation of Norelgestromin having an anti/syn Ratio of 1.97

Levonorgestrel (56.00 g; 0.1792 mol) was suspended in 840 ml of methanol. Sodium methoxide (10.65 g; 0.1971 mol; 1.10 eq) and then hydroxylamine hydrochloride (17.43 g; 0.2509 mol; 1.40 eq) were added to the suspension to form a mixture. The mixture was heated at 38° C. for 7½ hours and then was cooled at 5° C. Sodium methoxide (3.58 g; 0.0663 mol.; 0.37 eq) was added with 40 ml of methanol. The mixture was filtered keeping the temperature at 5° C. and the solid was washed with 80 ml of methanol. The filtrate was added dropwise to 1920 ml of cold water at a temperature of 0°-5° C. and kept at this temperature for 2 hours under stirring. The precipitate was recovered by filtration, washed with 1680 ml of water and dried under vacuum at 35° C. for 48 hours.

57.12 g of norelgestromin were obtained (yield 97.3%; HPLC purity 99.5%; HPLC anti/syn ratio 1.97)

### Example 2

### Preparation of Norelgestromin having an anti/syn Ratio of 1.37

Levonorgestrel (3.00 g; 9.60 mmol) was suspended in 60 ml of methanol. Sodium methoxide (0.259 g; mmol; 0.50 eq) and then hydroxylamine hydrochloride (0.934 g; 13.44 mmol; 1.40 eq) were added to the suspension to form a mixture. The mixture was heated at reflux for 3 hours. After 3 hours, the mixture was cooled at 5° C. Sodium methoxide (0.539 g; 9.98 mmol; 1.04 eq) was then added with 3 ml of methanol. The mixture was filtered keeping the temperature at 5° C. and the solid was washed with 6 ml of methanol. The filtrate was added dropwise to 138 ml of cold water at a temperature of 0°-5° C. and kept at this temperature for 2 hours under stirring. The precipitate was recovered by filtration, washed with 150 ml of water and dried under vacuum at 70° C. for 16 hours.

2.90 g of Norelgestromin were obtained (yield 92.3%; HPLC purity 96.9%, HPLC anti/syn ratio 1.37).

### Example 3

### Preparation of Norelgestromin having an anti/syn Ratio of 0.96

Levonorgestrel (1.00 g; 3.20 mmol) was suspended in 16 ml of methanol with hydroxylamine hydrochloride (0.311 g; 4.48 mmol; 1.40 eq). The mixture was refluxed for 4 hours and then the mixture was cooled at 5° C. Sodium methoxide (0.254 g; 4.70 mmol; 1.47 eq) was added with 2 ml of methanol. The mixture was filtered keeping the temperature at 5° C. and the filtrate was added dropwise to 36 ml of cold water at a temperature of 0°-5° C. and kept at this temperature for 2 hours under stirring. The precipitate was recovered by filtration, washed with 50 ml of water and dried under vacuum at 70° C. for 16 hours.

0.94 g of Norelgestromin was obtained (yield 89.7%; HPLC purity 95.1%, HPLC anti/syn ratio 0.96).

### Example 4

### Preparation of Norelgestromin having an anti/syn Ratio of 2.08

Levonorgestrel (30.00 g; 96.01 mmol) was suspended in 390 ml of methanol. Sodium methoxide (5.70 g; 105.6 mmol; 1.10 eq) and then hydroxylamine hydrochloride (9.34 g; 134.4 mmol; 1.40 eq) were added to the suspension to form a mixture. The mixture was heated at 46° C. for 16 hours and then was cooled at 5° C. Sodium methoxide (1.92 g; 35.52 mmol; 0.37 eq) was added with 15 ml of methanol. The mixture was filtered keeping the temperature at 5° C. and the solid washed with 60 ml of methanol. The filtrate was added dropwise to 930 ml of cold water at a temperature of 3-6° C. and kept at this temperature for 2 hours under stirring. The precipitate was recovered by filtration, washed with 300 ml of water and dried under vacuum at 35° C. for 22 hours.

29.21 g of norelgestromin were obtained (yield 92.9%; HPLC purity 99.7%; HPLC; anti/syn ratio 2.08).

### Example 5

### Preparation of Norelgestromin having an anti/syn Ratio of 2.08

Levonorgestrel (25.00 g; 80.01 mmol) was suspended in 250 ml of methanol. Sodium methoxide (4.75 g; 88.01 mmol; 1.10 eq) and then hydroxylamine hydrochloride (7.78 g; 112.01 mmol; 1.40 eq) were added to the suspension to form a mixture. The mixture was stirred at 20° C. for 16 hours. Sodium methoxide (1.92 g; 35.52 mmol; 0.37 eq) was added with 25 ml of methanol keeping the temperature below 20° C. The mixture was filtered and the solid was washed with 25 ml of methanol. The filtered solution was added dropwise to 600 ml of cold water at a temperature of 0-5° C. and kept at this temperature for 2 hours under stirring. The precipitate was recovered by filtration, washed with about 250 ml of water and dried under vacuum at 50° C. for 16 hours.

25.28 g of norelgestromin were obtained (yield 96.49%; HPLC purity 99.24%; HPLC; anti/syn ratio 1.92).

### Example 6

### Preparation of Norelgestromin having an anti/syn Ratio of 1.81

Levonorgestrel (7.00 g; 22.40 mmol.) was suspended in 112 ml of methanol. Sodium methoxide (1.33 g; 24.64 mmol.; 1.10 eq) and then hydroxylamine hydrochloride (2.18 g; 31.36 mmol.; 1.40 eq) were added to the suspension to form a mixture. The mixture was heated at 37° C. for 3½ hours and then was cooled at 5° C. Sodium methoxide (0.448 g; 8.29 mmol.; 0.37 eq) was added with 7 ml of methanol. The mixture was filtered keeping the temperature at 5° C., and the solid was washed with 7 ml of methanol. The filtrate was added dropwise to 252 ml of cold water at a temperature of 3-6° C. and kept at this temperature for 2 hours under stirring. The precipitate was recovered by filtration, washed with 252 ml of water and dried under vacuum at 35° C. for 24 hours.

6.60 g of norelgestromin were obtained (yield 89.9%; HPLC purity 99.73%; HPLC anti/syn ratio 1.81).

### Example 7

### Preparation of Norgestrimate having an anti/syn Ratio of 1.81

Levonorgestrel 17-acetate (10.00 g; 28.21 mmol) was suspended in 200 ml of methanol. Sodium methoxide (1.68 g; 31.03 mmol; 1.10 eq) and then hydroxylamine hydrochloride (2.74 g; 39.49 mmol; 1.40 eq) were added to the suspension to form a mixture. The mixture was heated at 40° C. for 3 hours and then was cooled at 5° C. Sodium methoxide (0.350 g; 6.49 mmol.; 0.23 eq) was added with 10 ml of methanol. The mixture was filtered keeping the temperature at 5° C. and the solid was washed with 20 ml of methanol. The filtrate was added dropwise to 460 ml of cold water at a temperature of t 0°-5° C. and kept at this temperature for 2 hours under stirring. The precipitate was recovered by filtration, washed with 100 ml of water and dried under vacuum at 40° C. for 48 hours.

10.17 g of norgestimate were obtained (yield 97.6%; HPLC purity 98.3%; HPLC anti/syn ratio 1.81)

HPLC

| Column: | Phenomenex Luna C18(2); 5 μm.; 150 × 4.6 mm. | |
|---|---|---|
| Mobile phase: | Eluent A: water/acetonitrile/tetrahydrofuran | |
| | 70:20:10 (v/v/v) | |
| | Eluent B: water/acetonitrile/tetrahydrofuran | |
| | 50:40:10 (v/v/v) | |
| Flow: | 1.5 ml/min | |
| Gradient of eluent: | Time (min) | Eluent B (%) |
| | 0.0 | 0.0 |
| | 30.0 | 0.0 |
| | 50.0 | 100.0 |
| | 80.0 | 100.0 |
| Stop time: | 80 min. | |
| Post time: | 10 min. | |
| Column temperature: | 60° C. | |
| Detector: | 243 nm. | |
| Injection volume: | 10 μl. | |

In this condition:

| | | Relative retention |
|---|---|---|
| | | time (RRT) |
| | Retention time (RT) | Vs. anti-isomer |
| Levonorgestrel | 19.7 | 0.76 |
| Norelgestromin syn-isomer | 23.6 | 0.90 |
| Norelgestromin anti-isomer | 26.1 | 1 |

What is claimed is:

1. A process for preparing a 3-oximino steroid of formula III 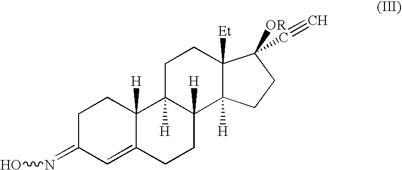
having an anti/syn ratio, of about 0.5 to about 3, comprising:
a) combining the corresponding precursor 3-oxosteroid of formula IV 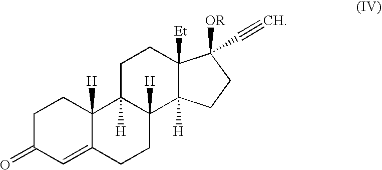
and a polar organic solvent to obtain a suspension;
b) combining the suspension of step (a) with a first base and a hydroxylammonium salt, to obtain a reaction mixture;
c) maintaining the obtained reaction mixture until the anti/syn ratio of the obtained 3-oximino steroid of the formula III is of about 0.5 to about 3;
d) combining a second base with the reaction mixture obtained in step c) when the desired anti/syn ratio of the obtained 3-oximino steroid is detected; and
e) recovering the compound of formula III,
wherein R is H or acetyl.

2. The process of claim 1, wherein R is H.

3. The process of claim 1, wherein R is acetyl.

4. The process of claim 1, wherein the 3-oximino steroid of the formula III has a purity of about 95% to about 100%, by HPLC.

5. The process of claim 2, wherein Norelgestromin has less than about 5% area by HPLC of Levonorgestrel.

6. The process of claim 5, wherein Norelgestromin has less than about 0.1% area by HPLC of Levonorgestrel.

7. The process of claim 3, wherein Norgestimate has less than about 5% area by HPLC of both Levonorgestrel 17-acetate and Norelgestromin.

8. The process of claim 7, wherein Norgestimate has less than about 2% area by HPLC of of both Levonorgestrel 17-acetate and Norelgestromin.

9. The process of claim 1, wherein the polar organic solvent in step (a) is selected from the group consisting of straight or branched C₁₋₄ alcohol, ether, nitrile, amide, and sulfoxide.

10. The process of claim 9, wherein the polar organic solvent in step (a) is selected from the group consisting of methanol, ethanol, 1-propanol, 2-propanol,t-butanol, 1,4-dioxane, acetonitrile, dimethylformamide, dimethylacetamide and dimethylsulfoxide.

11. The process of claim 10, wherein the solvent is methanol.

12. The process of claim 1, wherein the hydroxylammonium salt used in step (b) is selected from the group consisting of hydroxylamine hydrochloride, hydroxylamine sulphate and hydroxylamine phosphate.

13. The process of claim 12, wherein the hydroxylammonium salt is hydroxylamine hydrochloride.

14. The process of claim 1, wherein the hydroxylammonium salt is used in step (b) in an amount of about 1.4 mole equivalents per mole of Levonorgestrel or of Levonorgestrel 17-acetate.

15. The process of claim 1, wherein the first base used in step (b) is selected from the group consisting of alkoxide, alkali hydroxide, carbonate of alkali metals and acetate of alkali metal.

16. The process of claim 15, wherein said first base is selected from the group consisting of sodium methoxide, sodium ethoxide, sodium propoxide, sodium t-butoxide, potassium methoxide, potassium ethoxide, potassium propoxide, potassium t-butoxide, potassium hydroxide, sodium hydroxide, sodium acetate, potassium carbonate, sodium carbonate, potassium bicarbonate and sodium bicarbonate.

17. The process of claim 16, wherein said first base is sodium methoxide.

18. The process of claim 1, wherein the first base is used in step (b) in an amount of less than about 1.4 mole equivalents per mole equivalent of Levonorgestrel or of Levonorgestrel 17-acetate.

19. The process of claim 18, wherein said first base is used in an amount of about 1.1 mole equivalents per mole equivalent of Levonorgestrel or of Levonorgestrel 17-acetate.

20. The process of claim 1, wherein the first base is combined with the precursor 3-oxo steroid prior to the hydroxylammonium salt.

21. The process of claim 1, wherein the temperature in step (c) is of about 20° C. to about reflux.

22. The process of claim 1, wherein the mixture of step (c) is maintained for at least about 3 hours.

23. The process of claim 1, wherein the 3-oximino steroid prepared has an anti/syn ratio of about 2.08.

24. The process of claim 1, wherein the second base in step (d) is the same as the first base in step (b).

25. Norelgestromin containing less than about 5% area by HPLC of Levonorgestrel.

26. The norelgestromin of claim 25, containing less than about 0.1% area by HPLC of Levonorgestrel.

27. Noregestimate containing less than about 5% area by HPLC of both Levonorgestrel 17-acetate and Norelgestromin.

28. The noregestimate of claim 27, containing less than about 2% area by HPLC of both Levonorgestrel 17-acetate and Norelgestromin.
